(19) United States
(12) Patent Application Publication
(10) Pub. No.: US 2002/0086319 A1
(43) Pub. Date: Jul. 4, 2002

(54) Integrated device with surface-attached molecular moieties and related machine-readable information

(76) Inventors: Richard N. Ellson, Palo Alto, CA (US); James K. Foote, Cupertino, CA (US); Mitchell W. Mutz, Palo Alto, CA (US)

Correspondence Address: REED & ASSOCIATES, 800 MENLO AVENUE, SUITE 210, MENLO PARK, CA, 94025, US

(21) Appl. No.: 09/993,353

(22) Filed: Nov. 13, 2001

Related U.S. Application Data
(63) Continuation-in-part of application No. 09/712,818, filed on Nov. 13, 2000.

(51) Int. Cl. C12Q 1/68; G06F 19/00; G01N 33/48; G01N 33/50; G06F 17/60

(52) U.S. Cl. 435/6; 702/19; 705/40

(57) ABSTRACT

The invention provides a device comprising a substrate having a surface capable of attaching a plurality of molecular moieties, or a surface having a plurality of molecular moieties attached thereto. The substrate also contains machine-readable information relating to the molecular moieties. The information may be contained in a discrete region of the substrate that is non-coplanar with respect to the substrate surface having the plurality of molecular moieties attached thereto. The information may, for example, relate to the identity of the attached molecular moieties or to instructions for attaching the molecular moieties. Also provided are methods and machines for forming and using the devices.

### CROSS-REFERENCE TO RELATED APPLICATIONS

This is a continuation-in-part of U.S. patent application Ser. No. 09/712,818, filed Nov. 13, 2000, the disclosure of which is incorporated by reference herein.

### TECHNICAL FIELD

This invention relates generally to devices comprising a substrate having a plurality of surface-attached moieties and containing machine-readable information related thereto. More particularly, the invention relates to the formation and use of biomolecular arrays on a substrate in conjunction with machine-readable information contained within the same substrate.

### BACKGROUND

Extensive research in recent years has focused on the development and implementation of new methods and systems for evaluating potentially useful chemical compounds. In the biomacromolecule arena, for example, much recent research has been devoted to potential methods for rapidly and accurately identifying the properties of various oligomers of specific monomer sequences, including ligand and receptor interactions, by screening high density arrays of biopolymers including nucleotidic, peptidic and saccharidic polymers.

An ideal array preparation technique should provide for highly accurate deposition of minute volumes of fluids on a substrate surface, wherein droplet volume—and thus “spot” size on the substrate surface—can be carefully controlled and droplets can be precisely directed to particular sites on a substrate surface. Optimally, such a technique could be used with porous or even permeable surfaces, as such surfaces can provide substantially greater surface area on which to attach molecular moieties that serve as array elements, and would enable preparation of higher density arrays. One way in which such improved arrays may be formed involves the use of focused acoustic energy, as described in detail in U.S. Ser. No. 09/964,212 to Ellson, Foote and Mutz for “Acoustic Ejection of Fluids from a Plurality of Reservoirs,” filed Sep. 25, 2001 and assigned to Picoliter Inc. (Mountain View, Calif.). As explained in the aforementioned patent application, focused acoustic energy may be used to eject single fluid droplets from a free surface of a fluid (e.g., in a reservoir or well plate) toward designated sites on a substrate surface, enabling extraordinarily accurate and repeatable droplet deposition. This method allows biomolecular arrays to be formed in high yield, having densities similar to or better than those achievable using photolithographic or other techniques. Thus, this technology allows an array manufacturer to produce customized arrays to order for customers who provide the desired specifications.

With many types of arrays, manufacturers encounter difficulties with maintaining and managing the profusion of information related to the large number of molecular moieties within an array. For example, if one were to prepare an array containing 10⁶ different molecular moieties, the amount of information required to describe all of the components of the array would involve about two terabytes of data. Consequently, the memory required to store the product catalog would be enormous. Furthermore, the information describing an array could represent a customer's proprietary information. Hence, it would be beneficial to physically associate information relating to a customized array with the substrate on which the array itself would be attached, in order to ensure that access to the information would be restricted to authorized individuals.

There are a number of patents describing integrated devices that contain both surface-bound chemical moieties and related information in machine-readable format. For example, U.S. Pat. No. 6,030,581 to Virtanen describes an optical disk that is readable by a CD-ROM or DVD reader, wherein the disk has a first sector with a substantially self-contained assay means for reacting with an analyte and a second sector containing a control means for conducting the assay. As another example, U.S. Pat. No. 5,872,214 to Nova et al. describes a combination of a matrix with a memory means, wherein the matrix is made from materials similar to those used as supports in hybridization assays, and the memory means contains a data storage unit. As a further example, U.S. Pat. No. 5,935,786 to Reber et al. describes a support member having a first annular portion to support molecular receptors and a second annular portion to support machine-readable data that identifies each of the plurality of molecular receptors. Although these integrated devices have been described as useful in biomolecular analysis, particularly in automated assay applications, none of these patents discloses a customized array or means of formation thereof. In addition, the designs of some of these devices are not easily adapted for array formation and use. For example, the optical disks of U.S. Pat. No. 6,030,581 are asymmetrically weighted about the center of the disk, thereby requiring inertial compensation if one of these disks is to be rotated about its center. Furthermore, there is a coplanar spatial relationship between the software (i.e., machine-readable information) region of the disk and the sample preparation assay region. This coplanar relationship does not allow a protective layer to be easily applied to the assay portion (e.g., by spin coating) without interfering with the software region.

Thus, there is a need in the art for improved devices comprising a substrate having a plurality of surface-attached moieties and containing related machine-readable information that facilitates formation and/or use of those moieties, e.g., arrays. There is a corresponding need for a machine capable of reading, processing, and writing information associated with the substrate.

### SUMMARY OF THE INVENTION

Accordingly, it is an object of the present invention to provide devices and methods that overcome the above-mentioned disadvantages of the prior art. In one aspect of the invention, a device is provided comprising a substrate having a plurality of molecular moieties attached to the substrate surface and containing machine-readable information that relates to the attached molecular moieties. The machine-readable information is contained in a discrete region of the substrate, which is non-coplanar with respect to the substrate surface having the attached molecular moieties. The information may include, for example, the identity of a customer, secured information, shipping and/or billing information, the identity of at least one of the molecular moieties, information regarding the nature of attachment of the molecular moieties to the substrate surface, information relating to experimental conditions that describe potential uses of the molecular moieties, and/or information relating to the results of such experiments. The information may be electronically, magnetically, optically, and/or mechanically readable.

In another aspect, the invention relates to a device comprising a substrate having a surface adapted for attachment of a plurality of molecular moieties and containing machine-readable information relating to the attached moieties.

In still another aspect, the invention relates to the attachment of molecular moieties to the substrate surface of a device as described above. The method involves use of an apparatus comprising a reader for processing the machine-readable information and a means for attaching a plurality of molecular moieties to the surface of the substrate according to the machine-readable information.

In a further aspect, the invention relates to a method for using the molecular moieties according to instructions provided in the machine-readable information. The method involves use of an apparatus comprising a reader, as above, and a means for carrying out the method according to the instructions provided in the machine-readable information.

### BRIEF DESCRIPTION OF THE DRAWINGS

FIGS. 1A, 1B, and 1C, collectively referred to as FIG. 1, schematically illustrate a device of the present invention comprising a substrate in the form of a single disk.

FIG. 1A shows the top view of the substrate, and

FIG. 1B illustrates the cross-sectional view of the disk of FIG. 1A along dotted line A.

FIG. 1C shows a bottom view of the disk.

FIGS. 2A, 2B, 2C, and 2D, collectively referred to as FIG. 2, schematically illustrate another embodiment of the device, wherein the substrate comprises a cartridge containing a magnetic disk and having an exterior surface in the shape of a well plate.

FIG. 2A shows top view of the cartridge,

FIG. 2B illustrates the cross-sectional view of the cartridge of FIG. 2A along dotted line B, and

FIG. 2C illustrates the cross-sectional view of the cartridge of FIG. 2A along dotted line C.

FIG. 2D illustrates a bottom view of the cartridge.

FIG. 3 schematically illustrates in simplified cross-sectional view another embodiment of the inventive device in the form of a tape having two opposing surfaces, wherein molecular moieties are attached to one surface and a magnetic medium containing machine-readable information is attached to the opposing surface.

FIGS. 4A, 4B, and 4C, collectively referred to as FIG. 4, schematically illustrate in simplified cross-sectional view another embodiment of the inventive device in the form of a slide having two opposing surfaces, wherein molecular moieties are attached to one surface and a memory chip is embedded in the other surface.

FIG. 4A shows the top view of the slide, and

FIG. 4B illustrates the cross-sectional view of the slide of FIG. 4A along dotted line D.

FIG. 4C shows a bottom view of the slide.

FIGS. 5A, 5B, 5C, and 5D, collectively referred to as FIG. 5, illustrate a method wherein a dimer is synthesized in situ on the substrate of the device of FIG. 1.

FIG. 5A illustrates a machine spinning the substrate in order to read the machine-readable information contained in a spiral track of the substrate.

FIG. 5B illustrates the acoustic ejection of a droplet of a first fluid containing a first molecular moiety adapted for attachment to the surface of the substrate, which is adapted for attachment to the selected molecular moieties.

FIG. 5C illustrates the ejection of a droplet of a second fluid containing a second molecular moiety adapted for attachment to the first moiety.

FIG. 5D illustrates the substrate and the dimer synthesized in situ by the method illustrated in FIGS. 5A, 5B, and 5C.

### DETAILED DESCRIPTION OF THE INVENTION

Before describing the present invention in detail, it is to be understood that this invention is not limited to specific moieties, storage media, or device structures, as such may vary. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise. Thus, for example, reference to “a molecular moiety” includes a single molecular moiety as well as a plurality of moieties, reference to “an array” includes a single array as well as a plurality of arrays, reference to “a biomolecule” includes a single biomolecule as well as a plurality of biomolecules (that may be the same or different), and the like.

In describing and claiming the present invention, the following terminology will be used in accordance with the definitions set out below.

The terms “acoustic coupling” and “acoustically coupled” used herein refer to a state wherein an object is placed in direct or indirect contact with another object so as to allow acoustic radiation to be transferred between the objects without substantial loss of acoustic energy. When two entities are indirectly acoustically coupled, an “acoustic coupling medium” is needed to provide an intermediary through which acoustic radiation may be transmitted. Thus, an ejector may be directly acoustically coupled to a fluid, e.g., by immersing the ejector in the fluid or indirectly, by interposing an acoustic coupling medium between the ejector and the fluid to transfer acoustic radiation generated by the ejector through the acoustic coupling medium and into the fluid.

The term “adsorb” as used herein refers to the noncovalent retention of a molecule by a substrate surface. That is, adsorption occurs as a result of noncovalent interaction between a substrate surface and adsorbing moieties present on the molecule that is adsorbed. Adsorption may occur through hydrogen bonding, van der Waal's forces, polar attraction, or electrostatic forces (i.e., through ionic bonding). Examples of adsorbing moieties include, but are not limited to, amine groups, carboxylic acid moieties, hydroxyl groups, nitroso groups, sulfones, and the like. Often the substrate may be functionalized with adsorbent moieties to interact in a certain manner, as when the surface is functionalized with amino groups to render it positively charged in a pH neutral aqueous environment. Likewise, adsorbate moieties may be added in some cases to effect adsorption, as when a basic protein is fused with an acidic peptide sequence to render adsorbate moieties that can interact electrostatically with a positively charged adsorbent moiety.

The term “array” as used herein refers to a two-dimensional arrangement of features, such as an arrangement of reservoirs (e.g., wells in a well plate) or an arrangement of different materials, including ionic, metallic, or covalent crystalline, molecular crystalline, composite or ceramic, glassine, amorphous, fluidic, or molecular materials on a substrate surface (as in an oligonucleotide or peptidic array). Different materials in the context of molecular materials encompass chemical isomers (including constitutional, geometric, and stereoisomers) and, in the context of polymeric molecules, encompass constitutional isomers having different monomer sequences.

Arrays are generally comprised of regular, ordered features, as in, for example, a rectilinear grid, parallel stripes, spirals, and the like, but non-ordered arrays may also be advantageously used. An array is distinguished from the more general term “pattern” in that patterns do not necessarily contain regular and ordered features. The arrays or patterns formed using the devices and methods of the invention have no optical significance to the unaided human eye. For example, the invention does not involve ink printing on paper or other substrates in order to form letters, numbers, bar codes, figures, or other inscriptions that have optical significance to the unaided human eye. The arrays prepared using the method of the invention generally comprise in the range of about 4 to about 10,000,000 features and, more typically, about 4 to about 1,000,000 features.

The term “customized array” as used herein refers to an array formed or made to order according to specifications relating to the features of the array, e.g., composition, location, density, and morphology. A manufacturer may make a customized array for one or more external customers, or for internal use, in which case the manufacturer would itself be the customer. A customized array is typically, but not necessarily, produced on a substrate in low-volume production runs wherein no more than about 5000, preferably no more than about 500, more preferably no more than about 100, and optimally no more than about 1 substrate containing the same array is produced per production run.

The term “attached,” as in, for example, a substrate surface having a molecular moiety “attached” thereto, includes covalent binding, adsorption, and mechanical immobilization. The terms “binding” and “bound” are identical in meaning to the term “attached.”

The terms “biomolecule” and “biological molecule” are used interchangeably herein to refer to any organic molecule, whether naturally occurring, recombinantly produced, or chemically synthesized in whole or in part, that is, was, or can be a part of a living organism. The term encompasses, for example, nucleotides, amino acids, and monosaccharides, as well as oligomeric and polymeric species such as oligonucleotides and polynucleotides; peptidic molecules such as oligopeptides, polypeptides, and proteins; and saccharides such as disaccharides, oligosaccharides, polysaccharides, and the like. The term also encompasses ribosomes, enzyme cofactors, pharmacologically active agents, and the like.

It will be appreciated that, as used herein, the terms “nucleoside” and “nucleotide” refer to nucleosides and nucleotides that contain not only the conventional purine and pyrimidine bases, i.e., adenine (A), thymine (T), cytosine (C), guanine (G), and uracil (U), but also protected forms thereof, e.g., wherein the base is protected with a protecting group such as acetyl, difluoroacetyl, trifluoroacetyl, isobutyryl, or benzoyl, and purine and pyrimidine analogs. Suitable analogs are known to those skilled in the art and are described in the pertinent texts and literature. Common analogs include, but are not limited to, 1-methyladenine, 2-methyladenine, N⁶-methyladenine, N⁶-isopentyladenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromo-guanine, 8-chloroguanine, 8-aminoguanine, 8-methylguanine, 8-thioguanine, 5-fluoro-uracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methyl-aminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine, and 2,6-diaminopurine.

In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “oligonucleotide” shall be generic to polydeoxynucleotides (containing 2-deoxy-D-ribose), polyribonucleotides (containing D-ribose), any other type of polynucleotide that is an N-glycoside of a purine or pyrimidine base, and other polymers with non-nucleotidic backbones, provided that the polymers contain nucleobases in a configuration that allows for base pairing and base stacking, such as are found in DNA and RNA. Thus, these terms include known types of oligonucleotide modifications, for example, substitution of one or more of the naturally occurring nucleotides with an analog, internucleotide modifications such as, for example, those with uncharged linkages (e.g., methyl phosphonates, phosphotriesters, phosphoramidates, carbamates, etc.); with negatively charged linkages (e.g., phosphorothioates, phosphorodithioates, etc.); with positively charged linkages (e.g., aminoalklyphosphoramidates, aminoalkylphosphotriesters); containing pendant moieties, such as, for example, proteins (including nucleases, toxins, antibodies, signal peptides, poly-L-lysine, etc.); with intercalators (e.g., acridine, psoralen, etc.); and containing chelators (e.g., metals, radioactive metals, boron, oxidative metals, etc.). There is no intended distinction in length between the terms “polynucleotide” and “oligonucleotide,” and these terms will be used interchangeably. These terms refer only to the primary structure of the molecule. As used herein the symbols for nucleotides and polynucleotides are in accordance with the IUPAC-IUB Commission of Biochemical Nomenclature Recommendations (Biochemistry 9:4022, 1970).

The terms “peptide,” “peptidyl,” and “peptidic” as used throughout the specification and claims are intended to include any structure comprised of two or more amino acids. For the most part, the peptides in the present arrays comprise about 5 to about 10,000 amino acids, preferably about 5 to about 1000 amino acids. The amino acids forming all or part of a peptide may be any of the twenty conventional amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids in the peptidic molecules forming the present arrays may be replaced by a non-conventional amino acid. In general, conservative replacements are preferred. Conservative replacements substitute the original amino acid with a non-conventional amino acid that resembles the original in one or more of its characteristic properties (e.g., charge, hydrophobicity, and stearic bulk; for example, one may replace Val with Nval). The term “non-conventional amino acid” refers to amino acids other than conventional amino acids, and includes, for example, isomers and modifications of the conventional amino acids (e.g., D-amino acids), non-protein amino acids, post-translationally modified amino acids, enzymatically modified amino acids, constructs or structures designed to mimic amino acids (e.g., α,α-disubstituted amino acids, N-alkyl amino acids, lactic acid, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine), and peptides having the naturally occurring amide —CONH— linkage replaced at one or more sites within the peptide backbone with a non-conventional linkage such as N-substituted amide, ester, thioamide, retropeptide (—NHCO—), retrothioamide (—NHCS—), sulfonamido (—SO₂NH—), and/or peptoid (N-substituted glycine) linkages. Accordingly, the peptidic molecules of the array include pseudopeptides and peptidomimetics. The peptides of this invention can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

The term “discrete” is typically used herein in its ordinary sense and refers to a region of a substrate that constitutes a separate or distinct part with respect to another region of the substrate. Thus, one discrete region of a substrate, such as the interior region, is readily distinguishable from another region, such as the surface.

The terms “DVD” or “digital versatile disk” are interchangeably used herein and refer to a high-density compact disk for storing large amounts of data, such as those associated with high-resolution audio-visual material. More specifically, the term typically refers to an optical storage medium with improved capacity and bandwidth, as compared with CD-ROMs. DVDs are currently commercially available in both a single-layer format with a storage capacity of about 3.9 to about 4.7 gigabytes, and in a dual-layer format with a storage capacity of about 8.5 gigabytes. In addition, DVDs having a storage capacity of up to about 17 gigabytes or greater are known in the art.

The term “fluid” as used herein refers to matter that is nonsolid, or at least partially gaseous and/or liquid. A fluid may contain a solid that is minimally, partially, or fully solvated, dispersed, or suspended. Examples of fluids include, without limitation, aqueous liquids (including water per se and salt water) and nonaqueous liquids (such as organic solvents and the like). As used herein, the term “fluid” is not synonymous with the term “ink” in that an ink must contain a colorant and may not be gaseous.

The terms “focusing means” and “acoustic focusing means” refer to a means for causing acoustic waves to converge at a focal point, either by a device separate from the acoustic energy source that acts like an optical lens, or by the spatial arrangement of acoustic energy sources to effect convergence of acoustic energy at a focal point by constructive or destructive interference. A focusing means may be as simple as a solid member having a curved surface, or it may include complex structures such as those found in Fresnel lenses, which employ diffraction in order to direct acoustic radiation. Suitable focusing means also include phased array methods as are known in the art and described, for example, in U.S. Pat. No. 5,798,779 to Nakayasu et al., and by Amemiya et al. (1997) Proceedings of the 1997 IS&T NIP 13 International Conference on Digital Printing Technologies, pp. 698-702.

The term “hybridizing conditions” is intended to mean those conditions of time, temperature, pH, and the necessary amounts and concentrations of moieties and reagents sufficient to allow at least a portion of a nucleotidic moiety to anneal with its complementary sequence. As is well known in the art, the time, temperature, and pH conditions required to accomplish hybridization depend on the size or length of the oligonucleotide moiety to be hybridized, the degree of complementarity between the oligonucleotide probe and the target, and the presence of other materials in the hybridization reaction admixture. The actual conditions necessary for each hybridization step are well known in the art, or can be determined without undue experimentation.

The terms “library” and “combinatorial library” are used interchangeably herein to refer to a plurality of chemical or biological moieties present on the surface of a substrate, wherein each moiety is different from each of the other moieties. The moieties may be, for example, peptidic molecules and/or oligonucleotides.

The term “machine” as used herein refers to a device that produces an applied force or alters the magnitude and/or direction of an applied force in order to perform a task. For example, the term “machine” encompasses computers and other devices that can perform operations in order to read information from a substrate. Unless otherwise specified, the term “machine” does not encompass a human being.

The term “machine-readable information” as used herein refers to data, instructions, details, and other matter having a format that can be read by a machine. Typically, such information relates to surface-bound moieties, specifically to the formation, attachment, and/or use thereof. The information may be contained in a substrate having one or more types of information storage media, e.g., magnetic, optical, electronic, and/or mechanical. A CD-ROM (compact disk-read only memory) drive, for example, is a machine that can read optically encoded information contained in a CD-ROM. The term “additional information” as used herein refers to supplemental information that alters the overall significance of existing information. Additional information may be in the form of added data and/or deleted data, digital or otherwise.

The term “molecular moiety” refers to an intact molecule (including monomeric molecules, oligomeric molecules, and polymers), a molecular fragment, or a mixture of molecular moieties (as in, for example, an alloy or a laminate).

The term “near” is used herein to refer to the distance from the focal point of the focused acoustic radiation to the surface of the fluid from which a droplet is to be ejected. The distance should be such that the focused acoustic radiation directed into the fluid results in droplet ejection from the fluid surface, and that can be selected by one of ordinary skill in the art for any given fluid using straightforward and routine experimentation. Generally, however, a suitable distance between the focal point of the acoustic radiation and the fluid surface is in the range of about 1 to about 15 times the wavelength of sound in the fluid, more typically in the range of about 1 to about 10 times that wavelength, preferably in the range of about 1 to about 5 times that wavelength.

The term “non-coplanar” refers to the spatial relationship of two regions of an object wherein the regions are not on the same plane. For example, opposing surfaces of a flat member are considered non-coplanar. As another example, two adjoining square surfaces of a cube are non-coplanar.

“Optional” or “optionally” means that the subsequently described circumstance may or may not occur, so that the description includes instances where the circumstance occurs and instances where it does not.

The term “reservoir” as used herein refers to a receptacle or chamber for holding or containing a fluid. Thus, fluid in a reservoir necessarily has a free surface, i.e., a surface that allows a droplet to be ejected therefrom. A reservoir may also be a locus on a substrate surface within which a fluid is constrained.

The term “substantially” as in, for example, the phrase “substantially all molecules of an array,” refers to at least 90%, preferably at least 95%, more preferably at least 99%, and most preferably at least 99.9% of the molecules of an array. Other uses of the term “substantially” involve an analogous definition.

The term “substrate” as used herein refers to any material having a surface onto which one or more fluids may be deposited. The substrate may be constructed in any of a number of forms, such as disks, wafers, slides, well plates, and membranes, for example.

In addition, the substrate may be porous or nonporous as required for deposition of a particular fluid. Suitable substrate materials include, but are not limited to, supports that are typically used for solid phase chemical synthesis, e.g., polymeric materials (e.g., polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, divinylbenzene, and styrene-based polymers), agarose (e.g., Sepharose®), dextran (e.g., Sephadex®), cellulosic polymers and other polysaccharides, silica and silica-based materials, glass (particularly controlled pore glass, or “CPG”), functionalized glasses, ceramics, and such substrates treated with surface coatings, e.g., with microporous polymers (particularly cellulosic polymers such as nitrocellulose), microporous metallic compounds (particularly microporous aluminum), antibody-binding proteins (available from Pierce Chemical Co., Rockford Ill.), bisphenol A polycarbonate, or the like.

Substrates of particular interest are porous and, as identified above, include: uncoated porous glass slides, e.g., CPG slides; porous glass slides coated with a polymeric coating, e.g., an aminosilane or poly-L-lysine coating, thus having a porous polymeric surface; and nonporous glass slides coated with a porous coating. The porous coating may be a porous polymer coating, such as may be comprised of a cellulosic polymer (e.g., nitrocellulose) or polyacrylamide, or a porous metallic coating (for example, comprised of microporous aluminum). Examples of commercially available substrates having porous surfaces include Fluorescent Array Surface Technology (FAST™) slides available from Schleicher & Schuell, Inc. (Keene, N.H.), which are coated with a 10-30 μm thick porous, fluid-permeable nitrocellulose layer that substantially increases the available binding area per unit area of surface. Other commercially available porous substrates include CREATIVECHIP® permeable slides currently available from Eppendorf AG (Hamburg, Germany), and substrates having “three-dimensional” geometry, by virtue of an ordered, highly porous structure that enables reagents to flow into and penetrate through the pores and channels of the entire structure. Such substrates are available from Gene Logic, Inc. under the tradename “Flow-Thru Chip,” and are described by Steel et al. in Chapter 5 of Microarray Biochip Technology (BioTechniques Books, Natick, Mass., 2000).

The term “porous,” as in a “porous substrate” or a “substrate having a porous surface,” refers to a substrate or surface, respectively, having a porosity (void percentage) in the range of about 1% to about 99%, preferably about 5% to about 99%, more preferably in the range of about 15% to about 95%, and an average pore size of about 100 Å to about 1 mm, typically about 500 Å to about 0.5 mm.

The term “impermeable” is used in the conventional sense to mean not permitting water or other fluids to pass through. The term “permeable” as used herein means not impermeable. Thus, the terms “permeable substrate” and “substrate having a permeable surface” refer to a substrate or surface, respectively, that can be permeated with water or other fluids.

While the foregoing support materials are representative of conventionally used substrates, it is to be understood that the substrate may in fact comprise any biological, nonbiological, organic, and/or inorganic material, and may be in any of a variety of physical forms, e.g., particles, strands, precipitates, gels, sheets, tubing, spheres, containers, capillaries, pads, slices, films, plates, slides, and the like, and may further have any desired shape, such as a disk, square, sphere, circle, etc. The substrate surface may or may not be flat, e.g., the surface may contain raised or depressed regions. A substrate may additionally contain or be derivatized to contain reactive functionalities that covalently link a compound to the substrate surface. These are widely known and include, for example, silicon dioxide supports containing reactive Si—OH groups, polyacrylamide supports, polystyrene supports, polyethylene glycol supports, and the like. Moreover, a discrete portion of the substrate may be composed of data storage media for containing machine-readable information.

The term “surface modification” as used herein refers to the chemical and/or physical alteration of a surface by an additive or subtractive process to change one or more chemical and/or physical properties of a substrate surface or a selected site or region of a substrate surface. For example, surface modification may involve: (1) changing the wetting properties of a surface; (2) functionalizing a surface, i.e., providing, modifying, or substituting surface functional groups; (3) defunctionalizing a surface, i.e., removing surface functional groups; (4) otherwise altering the chemical composition of a surface, e.g., through etching; (5) increasing or decreasing surface roughness; (6) providing a coating on a surface, e.g., a coating that exhibits wetting properties that are different from the wetting properties of the surface; and/or (7) depositing particulates on a surface.

In one embodiment, then, the invention pertains to a device comprising a substrate having a plurality of molecular moieties attached to a surface thereof. The substrate also contains machine-readable information relating to the molecular moieties, wherein the information is contained in a discrete region of the substrate that is non-coplanar with respect to the substrate surface having the molecular moieties attached thereto. Preferably, the information is machine-readable and located on a surface that opposes the surface to which the molecular moieties are attached.

The machine-readable information may contain data for a single purpose or for multiple purposes. For example, the information may relate to a customized or a mass-manufactured array if the molecular moieties attached to the surface of the substrate form an array. With a customized array, the machine-readable information may identify a customer associated with the substrate. Alternatively, or in addition, the machine-readable information may include shipping and/or billing information. When appropriate, i.e., with confidential data, the machine-readable information may be in a secure form, e.g., as encrypted data, as password-protected data, or in some other form having restricted access. The machine-readable information may also include the identity of at least one of the molecular moieties attached to the device surface, a comprehensive description of one or more of the molecular moieties, information relating to the means by which the moieties are attached to the device surface, information relating to experimental conditions and procedures associated with one or more potential uses of the moieties, suggested or required storage conditions for the molecular moieties, and/or information relating to the results of an experiment associated with a use of the molecular moieties. When two or more of the devices are used simultaneously, e.g., in a single production run, information such as identification numbers, production lot number, and time stamps may be included.

Preferably, the machine-readable information is digital. Typically, the above-described machine-readable information in digital format requires at least about 1 kilobyte of data and sometimes at least about 1 megabyte of data. In certain instances, the machine-readable information may correspond to about 1 to about 650 megabytes of data. However, the information may indicate a simple matter such as whether a particular site has a molecular moiety attached thereto, in which case only one bit of data is needed to represent the information. In order to access the information with acceptable speed, the machine-readable information may conform to one or more readily readable formats. When the information is optically readable, a compact disk reader and/or a DVD reader may be used. In the alternative, or in addition, the optically readable information may be readable by a bar code reader, e.g., a one-dimensional or two-dimensional bar code reader. As a further alternative, the machine-readable information may be magnetically readable by any number of magnetic media readers, e.g., disk drives, tapes and ZIP® drives, that are known in the art. As a still further alternative, the machine-readable information may be electronically readable through the use of electrical contacts or an inductive reader. Optionally, the reader may transmit the read information to a remote site for processing. Providing additional human-readable information on the device may enhance ease of use, in which case, the device may further include magnetic media or optical media on which additional information may be written.

Typically, although not necessarily, the attached moieties are biomolecules. The biomolecules may be nucleotidic or peptidic, and monomeric, oligomeric, or polymeric. For use in assays and other sample analysis applications, automated or not, the plurality of attached moieties may form an array. It is envisioned that the inventive device may contain an array comprising at least about 50,000, preferably about 200,000, and optimally about 1,000,000 moieties per square centimeter of substrate surface. In order to protect the attached moieties from exposure to detrimental conditions, the inventive device may further comprise a protective layer over the attached moieties. Such a protective layer may or may not be removable from the attached moieties.

The device may have any of a number of different configurations. For example, the substrate of the device may comprise a disk, a tape, a well plate, or a slide. In some instances, the substrate may include a plurality of surfaces to which the molecular moieties may be attached, wherein the different surfaces are optionally arranged in a three-dimensional structure. Many well plates suitable for use as the substrate of the device are commercially available and may contain, for example, 96, 384, 1536 or 3456 wells per well plate. Manufacturers of such well plates include Corning Inc. (Corning, N.Y.) and Greiner America, Inc. (Lake Mary, Fla.). However, the availability of such commercially available well plates does not preclude manufacture and use of custom-made well plates containing at least about 10,000 wells, or as many as 100,000 wells or more. For array forming applications, it is expected that about 100,000 to about 4,000,000 reservoirs may be employed. In addition, it is preferable that the center of each reservoir be located not more than about 1 centimeter, preferably not more than about 1 millimeter, and optimally not more than about 0.5 millimeter from a neighboring reservoir center.

The specific substrate material is selected according to the required functionality of the substrate. For example, the material used must be compatible with the fluids with which the substrate may come into contact. Thus, when it is intended that the substrate come into contact with an organic solvent such as acetonitrile, polymers that dissolve or swell in acetonitrile would be unsuitable for use in forming the substrate. Similarly, for a substrate that may come into contact with dimethylsulfoxide, materials that are dimensionally unstable with respect to dimethylsulfoxide would be unsuitable.

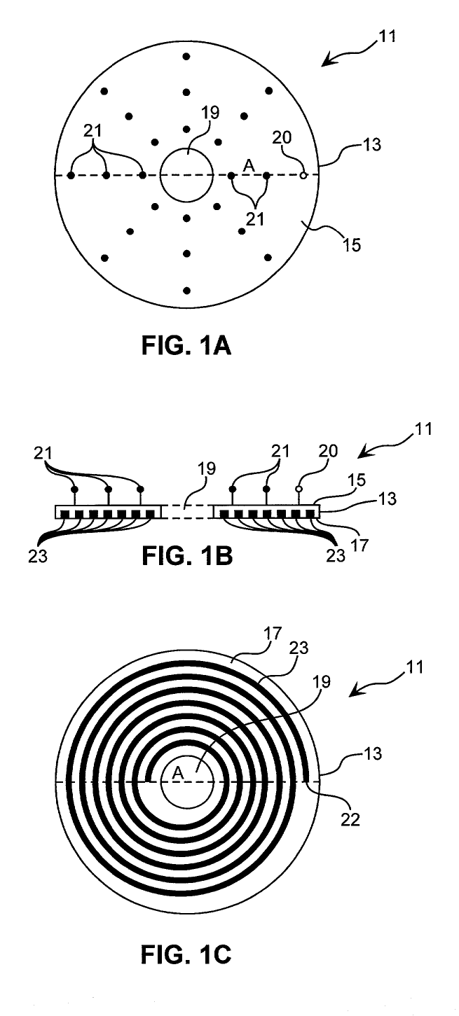

FIG. 1 schematically illustrates one embodiment of the inventive device wherein the substrate is in the form of a disk, specifically a compact disk. As with all figures referenced herein, in which like parts are referenced by like numerals, FIG. 1 is not necessarily to scale, and certain dimensions may be exaggerated for clarity of presentation. The device 11 is comprised of a solid circular disk 13 having opposing and substantially parallel surfaces, indicated at 15 and 17, respectively. Located at the center of the disk is a circular hole 19 extending through the disk. Attached to exterior surface 15 is a plurality of molecular moieties 21 in the form of an array. That is, the molecular moieties 21 represent features of the array, with the features forming concentric circles about the center hole 19 of the disk. As such, the disk is substantially symmetric about its center and is thus substantially rotationally uniform. Preferably, the radial mass distribution of the disk is also substantially uniform. Other distributions of moieties are possible provided that the mass distribution does not substantially interfere with the rotational stability of the disk. Rotational stability depends on mass distribution, rate of rotation, and other parameters of disk and rotational means design known in the art.

Information relating to the molecular moieties is shown contained in the disk 13 as a spiral track 23 of data encoded as a series of reflective features and non-reflective pits. The information is optically readable by rotating the disk 13 about the center hole 19 and using an optical reader to process (or “read”) the information from the underside 17 of the disk 13. Design and construction of such optical readers are well known in the art. As the information relating to the attached molecular moieties is located within the disk in a spiral track 23 rather than on the surface 15 to which the moieties 21 are attached, it is evident that the information is located in a discrete region of the disk that is non-coplanar with respect to surface 15 on which the moieties 21 are attached. Optionally, surface 15 may be covered with a protective layer (not shown) that reduces the risk of damage to the molecular moieties during handling.

When the substrate is symmetrical, axially or otherwise, it is useful to establish the orientation of the substrate with respect to a reader. Thus, either or both of surfaces 15 and 17 may be marked to establish proper orientation. For example, a reference moiety 20 may be used to establish a reference point on surface 15 such that the location of the reference moiety 20 corresponds to the location of the terminus 22 of the spiral track 23. As shown, molecular moiety 20 is located at the nearest point on surface 15 to the location of terminus 22. This allows the machine-readable information to act as a positional encoder for properly depositing the molecular moieties on the opposing surface. That is, the act of reading the machine-readable information from the spiral track 23 on surface 17 may determine the rotational position of the disk 13. This correspondence may be used to improve the timing of material release by a deposition system adapted for controlled delivery of materials to the substrate.

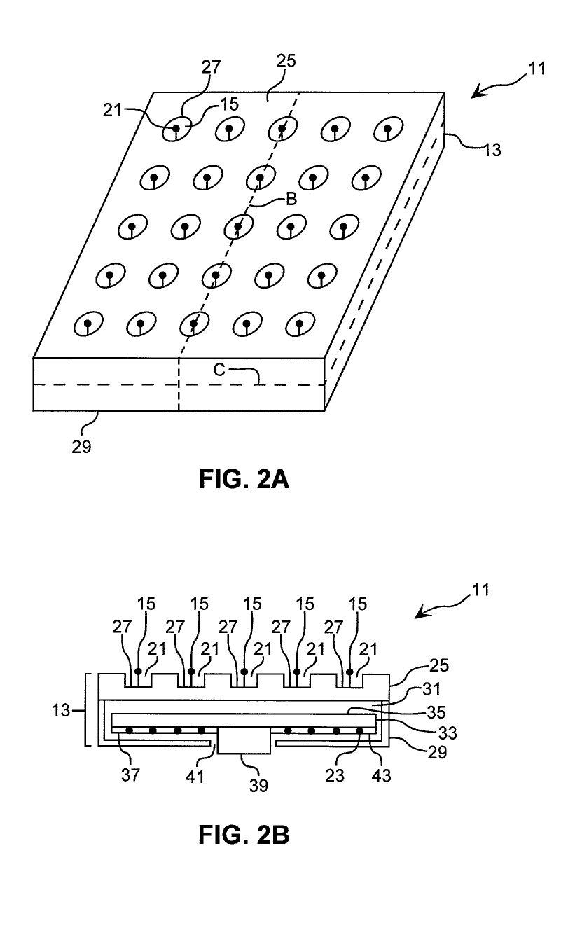

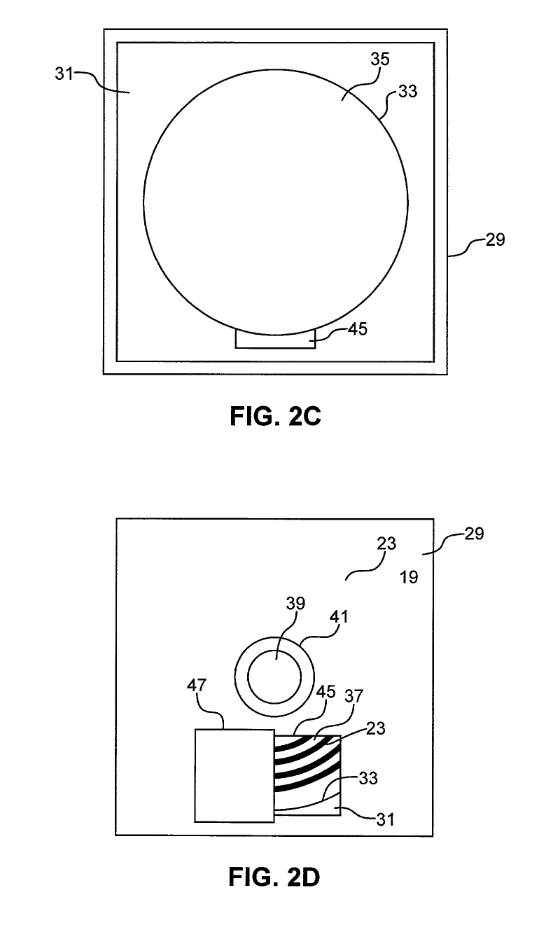

FIG. 2 schematically illustrates another embodiment of the inventive device wherein the substrate is in the form of a cartridge. The device 11 is comprised of a cartridge 13 having an upper region formed from a well plate 25 having individual wells 27 therein. Such well plates are commercially available from Corning Inc. (Corning, N.Y.) and Greiner America, Inc. (Lake Mary, Fla.). As shown, each individual well 27 has a molecular moiety 21 bound to an interior surface 15 thereof. The moiety, however, is not necessarily covalently bound to the plate. For example, the moiety may be in solution. As a general rule, though, if an array of moieties is located in an interior surface of the well, the array is bound to the surface. The well plate 25 is attached to a cartridge base 29 to define a cartridge interior 31. A magnetic disk 33 is generally interposed between well plate 25 and the cartridge base 29 within the cartridge interior 31. The disk 33 is a generally flat and circular piece having an upper surface 35 and a lower surface 37. A cylindrical hub 39 extends perpendicularly from the center of the lower surface 37 of the disk 33 through circular opening 41 of the cartridge base 29. The disk is free to rotate about its hub in a generally free-floating manner. The lower surface 37 is coated with magnetic storage medium 43 that allows a spiral track 23 to be formed therein to magnetically store machine-readable information related to the moieties.

Also located in the cartridge base 29 is a rectangular opening 45 that provides external access to the magnetic disk contained in the cartridge interior 31. A slidable spring-loaded panel 47 covers the opening 45 in order to protect the magnetic medium on the disk from damage when the disk is not in use. As shown, the slidable panel is positioned such that it does not cover the opening, thereby providing a magnetic reader access to the magnetic medium on the disk. Thus, the information contained in the spiral track 23 is ready for reading by a magnetic reader. Design, construction, and use of such magnetic readers are well known in the art. For example, the magnetic reader may engage the disk by gripping the portion of the hub 39 that is accessible to the exterior to the cartridge and spinning the disk. This allows information contained in the spiral track to be read. As the information relating to the attached moieties is located within the disk in a spiral track 23 rather than on the interior surfaces 15 of the well plate to which the moieties 21 are attached, it is evident that the information is located in a discrete region of the disk that is non-coplanar with respect to the interior surfaces 15. Optionally, one or more of the interior surfaces 15 may be covered with a protective layer (not shown) that protects the moieties from damage as a result of improper handling. Devices for sealing well plates are commercially available from many sources including TekCel Corporation (Hopkinton, Mass.).

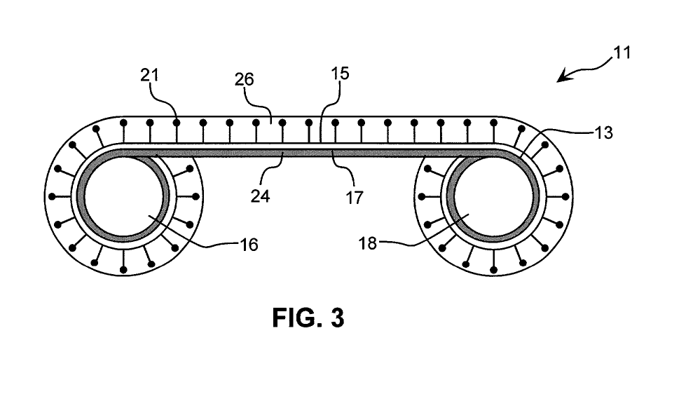

FIG. 3 schematically illustrates in simplified cross-sectional view another embodiment of a device of the invention in the form of a tape. The tape 13 has opposing and substantially parallel surfaces, indicated at 15 and 17, respectively. The tape is shown as a web under tension and extending between two spools, indicated at 16 and 18 respectively, wherein upper surface 15 faces outward from each spool. Attached to surface 15 is a plurality of molecular moieties 21 in the form of an array. That is, the molecular moieties 21 represent features of the array and each feature is equidistant from its nearest neighboring feature. A protective layer 26 encases the moieties to protect them from damage as the spools wind and unwind the tape. As an alternative (not shown), the protective layer may be provided as a spacer that does not encase the moieties, but rather forms wells in combination with the tape, each well containing a moiety. In such a case, the spacer would prevent the lower surface of the tape from contacting the moieties attached to the upper surface of the tape, thus preventing damage when the tape is spooled.

Located on the lower surface 17 of the tape 13 is a layer of magnetic data storage medium 24 containing machine-readable information relating to the attached molecular moieties. The information is contained in the magnetic medium 24 as a linear track of data that is preferably digital but may be analog if desired. The information is magnetically readable by passing the tape over a magnetic reader adapted to read the information from surface 17 of the tape 13. As the information relating to the attached moieties is located within the tape as a linear track rather than on the surface 15 on which the moieties 21 are attached, it is evident that the information is located in a discrete region of the tape that is non-coplanar with respect to surface 15.

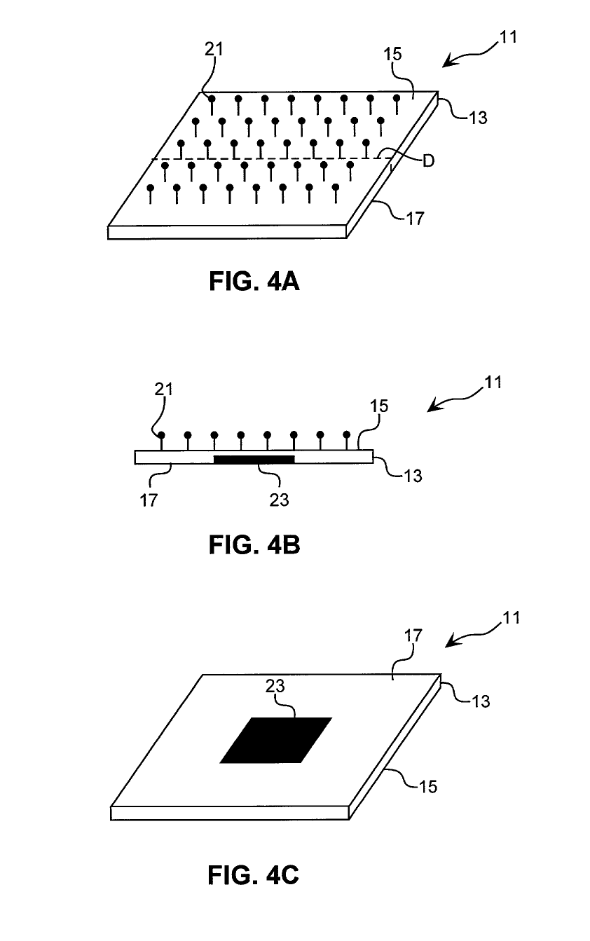

FIG. 4 schematically illustrates in simplified cross-sectional view another embodiment of the inventive device in which the substrate is an ordinary microscope slide. That is, the device 11 is comprised of a rectangular slide 13 having opposing and substantially parallel surfaces, indicated at 15 and 17, respectively. The slide may be of any convenient size, but is preferably a standard glass microscope slide that has a rectangular surface of about 3 inches by 1 inch (75 mm×25 mm). Optionally, the slide may have coatings of substantially uniform thickness applied to various regions of one surface to form a raised exterior surface. Attached to exterior surface 15 is a plurality of molecular moieties 21 in the form of an array. That is, the molecular moieties 21 represent individual features of the array, with the features forming a preferably rectilinear array such that each feature has four nearest neighbors, each equidistant from the first.

Information relating to the molecular moieties is contained in an electronic microchip 23 that provides sufficient memory to store such information. As shown, the microchip 23 is embedded in the slide 13. Such a microchip 23 may be partially exposed at surface 17, as shown in FIG. 4, or may be located entirely within the substrate. Such microchips are often employed in “smart cards,” i.e., plastic cards resembling a credit card that contains a computer chip, which enables the holder to perform various operations, such as mathematical calculations, paying of bills, and the purchasing of goods and services. There are many commercial sources of smart cards and smart-card readers, one of which is Gemplus Corporation (Redwood City, Calif.).

In operation, such chips may be read through electrical or physical contact with a reader or by using a contactless card reader that accesses data in the card through a radio frequency signal or through magnetic induction. Moreover, certain microchips have been designed for use in cards that are able to withstand temperatures up to about 90° C. without visual or functional alteration. Such microchips are particularly useful for devices that may have to withstand high temperatures for an extended period of time. For example, the inventive device may contain attached oligonucleotides that serve as probes to assess whether target nucleotidic moieties are present in a sample. Typical hybridization conditions for relatively short oligonucleotides, about 2 to about 20 nucleotides in length, involve temperatures of about 30° C. to about 50° C., optimally about 35° C. to about 45° C. However, longer oligonucleotides generally require higher hybridization temperatures, e.g., about 50° C. to about 80° C., in order to provide completion of hybridization to an acceptable extent within an acceptable amount of time. Thus, for any of the embodiments of the invention, it is desirable for all components of the substrate, including, for example, the regions containing the machine-readable information, to be able to withstand the conditions associated with the attachment and/or use of the moieties to the substrate. These conditions include, for example, temperature, pressure, and humidity. It is well known that most silicate glasses and certain polymers, such as perfluorinated polyalkenes, polyesters, and polyimides, are typically dimensionally stable at ordinary hybridization conditions.

Thus, smart card technology as described above represents an aspect of another embodiment of the invention in which the data associated with the machine-readable information are stored in a data storage medium that is sufficiently robust to survive exposure to the test conditions of the attached molecular moieties. That is, the machine-readable information is contained in a discrete region of the substrate that does not degrade when the substrate is exposed to test conditions associated with the moieties. Even after exposure to test conditions, the machine-readable information is still intact and machine-readable. In such a case, the machine-readable information and the attached molecular moieties may be later positioned in coplanar relationship with each other. For example, the machine-readable information may be stored on a microscope slide as fluorescent spots of varying intensities. Such information may be read and digitized by the same fluorescence reader used to measure the degree of hybridization in a moiety binding experiment. Data encoding methods for storing digital data as analog intensities are well known to those skilled in the art. Some fluorescence readers, such as the GenePix 4000 from Axon Instruments, Inc. (Foster City, Calif.) have an additional advantage as readers of analog light intensities as they can scan simultaneously for more than one fluorescent frequency. This would enable inclusion of more than one data channel in the same location on the slide. Similarly, nonfluorescent signals, e.g., magnetic or radioactive signals, may be used to represent machine-readable information and to indicate to a condition associated with the attached moieties.

The invention represents a substantial improvement in the art for a number of reasons. As an initial matter, it is advantageous to physically associate machine-readable information relating to substrate-bound molecular moieties with the moieties themselves to prevent mislabeling. In addition, devices for attaching molecular moieties to a substrate surface and/or for performing experiments with the moieties will not generally be suitable for reading machine-readable data. Thus, by providing machine-readable information and the surface-bound moieties in discrete regions of the device, a machine may be designed as a combination of two separate devices, one to manipulate (i.e., to attach, modify or otherwise make use of) the molecular moieties and the other to read information. Furthermore, by providing the molecular moieties and the machine-readable information on non-coplanar surface segments, the device allows a protective layer to be formed over the molecular moieties yet does not prevent access to the machine-readable information. For example, a UV-transparent protective layer could be used if measurement of UV emission from the attached molecular moieties were desired. As another example, the protective layer may provide the moieties access to a reactant but not to other matter. In short, the non-coplanar aspect of the device allows for greater flexibility in the design of machines for use with the device. Other advantages may become apparent through use of, or routine experimentation with, the device.

Attachment molecular moieties may be accomplished by using any suitable device for attaching compounds, molecular fragments, or molecular mixtures to a substrate surface. Such a device enables preparation to order of molecular arrays, particularly biomolecular arrays, having densities allowed by the array-producing technology, such as photolithographic processes, piezoelectric techniques (e.g., using inkjet printing technology), and microspotting. A preferred device is described in U.S. patent application Ser. No. 09/669,996, cited supra. When focused acoustic energy is used, as described in the '996 patent application, the array densities that may be achieved using the devices and methods of the invention are at least about 50,000 biomolecules per square centimeter of substrate surface, preferably at least about 200,000 per square centimeter of substrate surface. The biomolecules may be, for example, peptidic molecules and/or oligonucleotides. The device may also comprise a means for altering the information contained in, adding information to, or deleting information from, the substrate.

The above-described device or another device may be used to carry out a method for attaching the plurality of molecular moieties to a surface of a substrate. Information may be read from the substrate, and a plurality of moieties may be attached to a surface of the substrate based upon the information. The reading step may involve moving the substrate with respect to the machine, wherein the moving step involves rotating or laterally moving the substrate. In the alternative or in addition, the reading step may comprise converting the information contained in the substrate into electric current or converting the information contained in the substrate into light waves.

The attaching step may comprise the step of ejecting fluid droplets onto the surface. The ejecting step may be carried out acoustically, with or without a nozzle. The attachment may comprise attaching no more than one molecular moiety at a time. In the alternative or in addition, the attaching step may comprise using a photolithographic technique. The attaching step may also comprise lowering the temperature of the substrate. The moieties may be covalently or noncovalently attached to the surface. Preferably, the machine that reads information from the substrate performs the attaching step. When this is the case, the machine may perform the reading and attaching steps separately, simultaneously, or alternating repeatedly until all attachment is completed.

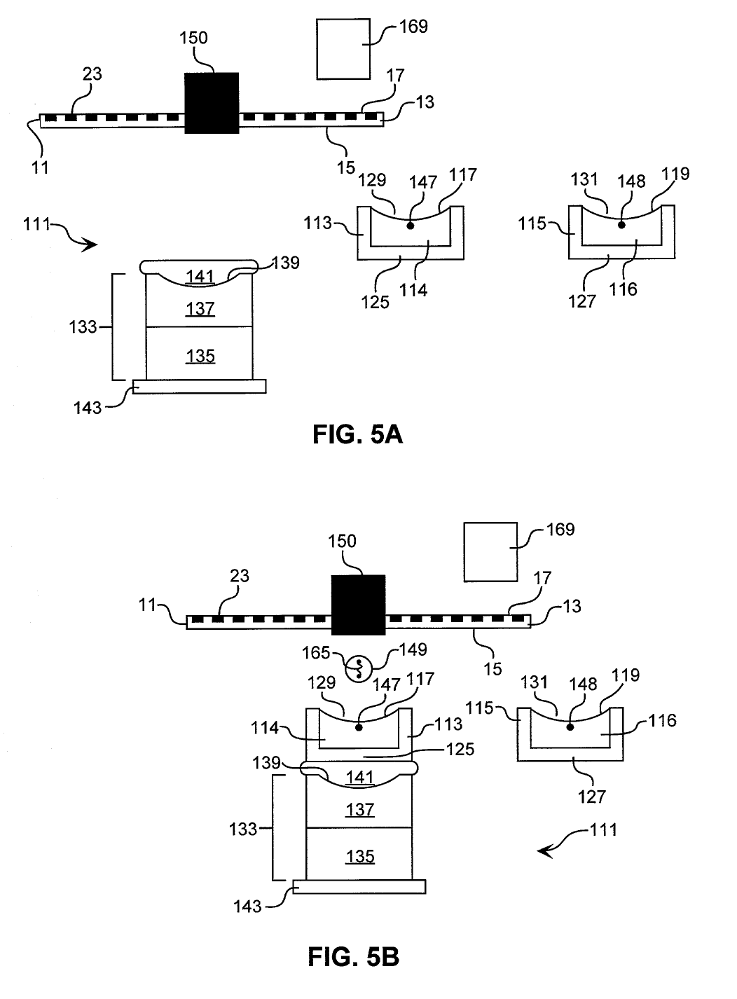

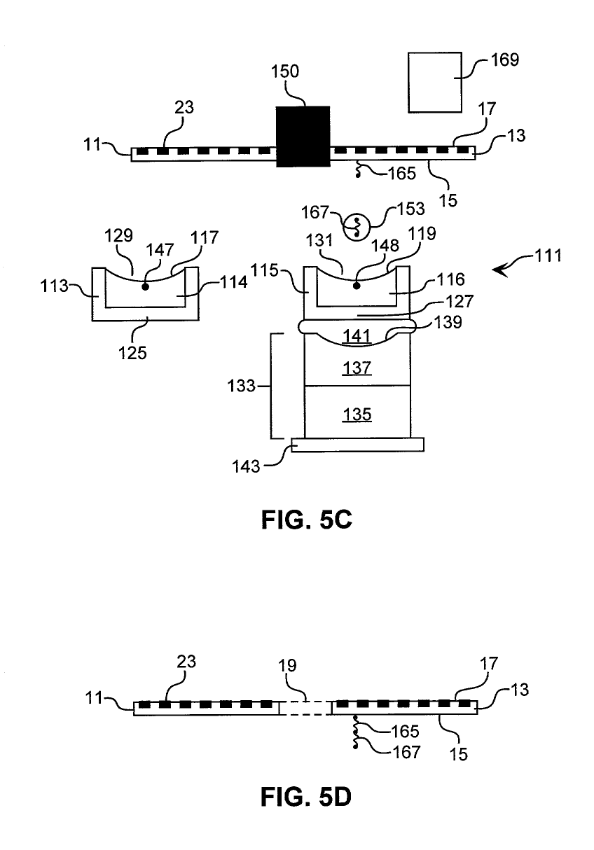

This method is useful in a number of applications including, but not limited to, spotting oligomers to form an array on a substrate surface or synthesizing array oligomers in situ. FIG. 5 schematically illustrates in simplified cross-sectional view a specific embodiment of the aforementioned method in which a dimer is synthesized on a substrate using a machine. It is important to note that other machines may be used for such synthesis or for attaching a plurality of moieties to a surface. The machine uses focused acoustic energy in order to eject fluids from a surface. The machine includes a plurality of reservoirs, i.e., at least two reservoirs. For simplicity, the machine 111 is illustrated as containing two reservoirs, with a first reservoir indicated at 113 and a second reservoir indicated at 115. The first fluid 114 within the first reservoir 113 has a first surface 117, and the second fluid 116 in the second reservoir 115 has a second surface 119. Each reservoir contains a fluid and the individual fluids in the different reservoirs may be the same or different. As shown, the reservoirs are of substantially identical construction so as to be substantially acoustically indistinguishable, but identical construction is not a requirement. The reservoirs are shown as separate removable components but may, if desired, be fixed within a plate or other substrate. For example, the plurality of reservoirs may comprise individual wells in a well plate, optimally although not necessarily arranged in an array. Each of the reservoirs 113 and 115 is preferably axially symmetric as shown, having vertical walls 121 and 123 extending upward from circular reservoir bases 125 and 127 and terminating at openings 129 and 131, respectively, although other reservoir shapes may be used. The material and thickness of each reservoir base should be such that acoustic radiation may be transmitted therethrough and into the fluid contained within the reservoirs.

The machine also includes an acoustic ejector 133 comprised of an acoustic radiation generator 135 for generating acoustic radiation and a focusing means 137 for focusing the acoustic radiation at a focal point within the fluid from which a droplet is to be ejected, near the fluid surface. As shown, the focusing means 137 may comprise a single solid piece having a concave surface 139 for focusing acoustic radiation, but may be constructed in other ways as discussed below. The acoustic ejector 133 is thus adapted to generate and focus acoustic radiation so as to eject a droplet of fluid from each of the fluid surfaces 117 and 119 when acoustically coupled to reservoirs 113 and 115, respectively. The acoustic radiation generator 135 and the focusing means 137 may function as a single unit controlled by a single controller, or they may be independently controlled, depending on the desired performance of the machine. Typically, single ejector designs are preferred over multiple ejector designs because accuracy of droplet placement and consistency in droplet size and velocity are more easily achieved with a single ejector.

As will be appreciated by those skilled in the art, any of a variety of focusing means may be employed in conjunction with the present invention. For example, one or more curved surfaces may be used to direct acoustic radiation to a focal point near a fluid surface. One such technique is described in U.S. Pat. No. 4,308,547 to Lovelady et al. Focusing means with a curved surface have been incorporated into the construction of commercially available acoustic transducers such as those manufactured by Panametrics Inc. (Waltham, Mass.). In addition, Fresnel lenses are known in the art for directing acoustic energy at a predetermined focal distance from an object plane. See, e.g., U.S. Pat. No. 5,041,849 to Quate et al. Fresnel lenses may have a radial phase profile that diffracts a substantial portion of acoustic energy into a predetermined diffraction order at diffraction angles that vary radially with respect to the lens. The diffraction angles should be selected to focus the acoustic energy within the diffraction order on a desired object plane.

There are also a number of ways to acoustically couple the ejector 133 to each individual reservoir and thus to the fluid therein. One such approach is through direct contact as is described, for example, in U.S. Pat. No. 4,308,547 to Lovelady et al., wherein a focusing means constructed from a hemispherical crystal having segmented electrodes is submerged in a liquid to be ejected. This patent further discloses that the focusing means may be positioned so as to provide a focal point at or below the surface of the liquid. This approach for acoustically coupling the focusing means to a fluid is, however, undesirable when the ejector is used to eject different fluids in a plurality of containers or reservoirs, as repeated cleaning of the focusing means would be required in order to avoid cross-contamination. The cleaning process would necessarily lengthen the transition time between each droplet ejection event. In addition, in such a method, fluid would adhere to the ejector as it is removed from each container, wasting material that may be costly or rare.

Optimally, acoustic coupling is achieved between the ejector and each of the reservoirs through indirect contact, as illustrated in FIG. 5A. In the figure, an acoustic coupling medium 141 is placed between the ejector 133 and the base 135 of reservoir 113, with the ejector and reservoir located at a predetermined distance from each other. The acoustic coupling medium may be an acoustic coupling fluid, preferably an acoustically homogeneous material in conformal contact with both the acoustic focusing means 137 and each reservoir. In addition, it is important to ensure that the fluid medium is substantially free of material having different acoustic properties than the fluid medium itself. As shown, the first reservoir 113 is acoustically coupled to the acoustic focusing means 137 such that an acoustic wave is generated by the acoustic radiation generator and directed by the focusing means 137 into the acoustic coupling medium 141, which then transmits the acoustic radiation into the reservoir 113.

The machine also comprises an optical reader 169 that reads the machine-readable information on the device 11. As shown in FIG. 5A, a substrate positioning means 150 of the machine engages the disk 13, which is rotated about its center. The optical reader 169 produces a collimated beam of light, which is directed to the spiral track 23 on the disk 13 to read the machine-readable information contained therein. Once the information is read, as shown in FIG. 5B, the disk 13 is positioned by the substrate positioning means 150 such that surface adapted for attachment to moieties is located directly over reservoir 113. FIG. 5B also shows that the ejector 133 is positioned by the ejector positioning means below reservoir 113 to acoustically couple the ejector and the reservoir through acoustic coupling medium 141. Once properly aligned, the ejector 133 is activated so as to eject droplet 149 onto the substrate 13. Droplet 149 contains a first monomeric moiety 165, preferably a biomolecule such as a protected nucleoside or amino acid, which after contact with the substrate surface attaches thereto by covalent bonding or adsorption.

Then, as shown in FIG. 5C, the disk 13 is again repositioned by the substrate positioning means 150 such that the site having the first monomeric moiety 165 attached thereto is located directly over reservoir 115 in order to receive a droplet therefrom. FIG. 5B also shows that the ejector 133 is positioned by the ejector positioning means below reservoir 115 to acoustically couple the ejector and the reservoir through acoustic coupling medium 141. Once properly aligned, the ejector 133 is again activated so as to eject droplet 153 onto the substrate 13. Droplet 153 contains a second monomeric moiety 167, adapted for attachment to the first monomeric moiety 165, typically involving formation of a covalent bond so as to generate a dimer as illustrated in FIG. 5D. The aforementioned steps may be repeated to generate an oligomer, e.g., an oligonucleotide, of a desired length and sequence based upon the machine-readable information contained in the substrate of the device.

Depending on the desired moieties to be attached, the machine may be adapted to eject fluids of virtually any type and amount desired. The fluid may be aqueous and/or nonaqueous. Examples of fluids include, but are not limited to, aqueous fluids including water per se, water-solvated ionic and non-ionic solutions, organic solvents, lipidic liquids, suspensions of immiscible fluids, and suspensions or slurries of solids in liquids. Because the invention is readily adapted for use with high temperatures, fluids such as liquid metals, ceramic materials, and glasses may be used; see, e.g., co-pending patent application U.S. Ser. No. 09/669,194 (“Method and Apparatus for Generating Droplets of Immiscible Fluids”), inventors Ellson and Mutz, filed on Sep. 25, 2000, and assigned to Picoliter, Inc. (Mountain View, Calif.). U.S. Pat. Nos. 5,520,715 and 5,722,479 to Oeftering describe the use of acoustic ejection for liquid metal for forming structures using a single reservoir and adding fluid to maintain focus. U.S. Pat. No. 6,007,183 to Horine is another patent that pertains to the use of acoustic energy to eject droplets of liquid metal. The capability of producing fine droplets of such materials is in sharp contrast to piezoelectric technology, insofar as piezoelectric systems perform suboptimally at elevated temperatures. Furthermore, because of the precision that is possible using the inventive technology, the machine may be used to eject droplets from a reservoir adapted to contain no more than about 100 nanoliters of fluid, preferably no more than 10 nanoliters of fluid. In certain cases, the ejector may be adapted to eject a droplet from a reservoir adapted to contain about 1 to about 100 nanoliters of fluid. This is particularly useful when the fluid to be ejected contains rare or expensive biomolecules, wherein it may be desirable to eject droplets having a volume of about 1 picoliter or less, e.g., having a volume in the range of about 0.025 pL to about 1 pL.

It will be appreciated that various components of the machine may require individual control or synchronization to form an array on a substrate. For example, the ejector positioning means may be adapted to eject droplets from each reservoir in a predetermined sequence associated with an array to be prepared on a substrate surface. Similarly, the substrate positioning means for positioning the substrate surface with respect to the ejector may be adapted to position the substrate surface to receive droplets in a pattern or array thereon. Either or both positioning means, i.e., the ejector positioning means and the substrate positioning means, may be constructed from, for example, motors, levers, pulleys, gears, a combination thereof, or other electromechanical or mechanical means known to one of ordinary skill in the art. It is preferable to ensure that there is a correspondence between the movement of the substrate, the movement of the ejector, and the activation of the ejector to ensure proper array formation.

It should be apparent to one of ordinary skill in the art that other steps may be required in order to perform oligomeric or polymeric synthesis/attachment. The above description is intended only as a simplified example. In addition, the chemistry employed in synthesizing substrate-bound oligonucleotides in this way will generally involve now-conventional techniques known to those skilled in the art of nucleic acid chemistry and/or described in the pertinent literature and texts. See, for example, DNA Microarrays: A Practical Approach, M. Schena, Ed. (Oxford University Press, 1999). That is, the individual coupling reactions may be conducted under standard conditions used for the synthesis of oligonucleotides and conventionally performed with automated oligonucleotide synthesizers. Such methodology is described, for example, in D. M. Matteuci et al. (1980) Tet. Lett. 521:719, in U.S. Pat. No. 4,500,707 to Caruthers et al., and in U.S. Pat. Nos. 5,436,327 and 5,700,637 to Southern et al.

Alternatively, an oligomer may be synthesized prior to attachment to the substrate surface and then “spotted” onto a particular locus on the surface using the methodology of the invention as described in detail above. Again, the oligomer may be an oligonucleotide, an oligopeptide, or any other biomolecular (or nonbiomolecular) oligomer moiety. Preparation of substrate-bound peptidic molecules, e.g., in the formation of peptide arrays and protein arrays, is described in co-pending patent application U.S. Ser. No. 09/669,997 (“Focused Acoustic Energy in the Preparation of Peptidic Arrays”), inventors Mutz and Ellson, filed on Sep. 25, 2000 and assigned to Picoliter, Inc. (Mountain View, Calif.). Preparation of substrate-bound oligonucleotides, particularly arrays of oligonucleotides wherein at least one of the oligonucleotides contains partially nonhybridizing segments, is described in co-pending patent application U.S. Ser. No. 09/669,267 (“Arrays of Oligonucleotides Containing Nonhybridizing Segments”), inventor Ellson, also filed on Sep. 25, 2000 and assigned to Picoliter, Inc. (Mountain View, Calif.).

Depending on the types of moieties to be attached to the substrate surface, a substrate surface may be modified prior to formation of a pattern or an array of the moieties thereon. Surface modification may involve functionalization or defunctionalization, smoothing or roughening, changing surface conductivity, coating, degradation, passivation, or otherwise altering the surface's chemical composition or physical properties. A preferred surface modification method involves altering the wetting properties of the surface, for example to facilitate confinement of a droplet ejected onto the surface within a designated area or enhancement of the kinetics for the surface attachment of molecular moieties contained in the ejected droplet. A preferred method for altering the wetting properties of the substrate surface involves deposition of droplets of a suitable surface modification fluid at each designated site of the substrate surface prior to acoustic ejection of fluids to form an array thereon. In this way, the “spread” of the acoustically ejected droplets may be optimized and consistency in spot size (i.e., diameter, height and overall shape) ensured. One way to implement the method involves acoustically coupling the ejector to a modifier reservoir containing a surface modification fluid and then activating the ejector, as described in detail above, to produce and eject a droplet of surface modification fluid toward a designated site on the substrate surface. The method is repeated as desired to deposit surface modification fluid at additional designated sites. This method is useful in a number of applications including, but not limited to, spotting oligomers to form an array on a substrate surface or synthesizing array oligomers in situ. As noted above, other physical properties of the surface that may be modified include thermal properties and electrical conductivity.

Certain performance-enhancing means may be provided to enhance moiety substrate surface attachment. For example, the machine may include a cooling means for lowering the temperature of the substrate surface to ensure, for example, that the ejected droplets adhere and become attached, wholly or partially, to the substrate. The cooling means may be adapted to maintain the substrate surface at a temperature that allows fluid to partially, or preferably substantially, solidify after the fluid comes into contact therewith. In the case of aqueous fluids, the cooling means should have the capacity to maintain the substrate surface at about 0° C. In addition, repeated application of acoustic energy to a reservoir of fluid may result in heating of the fluid. Heating can, of course, result in unwanted changes in fluid properties such as viscosity, surface tension, and density. Thus, the machine may further comprise means for maintaining fluid in the reservoirs at a constant temperature. Design and construction of such temperature maintaining means are known to one of ordinary skill in the art and may comprise, e.g., components such a heating element, a cooling element, or a combination thereof. For many biomolecular deposition applications, it is generally desired that the fluid containing the biomolecule be kept at a constant temperature without deviating more than about 1° C. or 2° C. therefrom. In addition, for a biomolecular fluid that is particularly heat sensitive, it is preferred that the fluid be kept at a temperature that does not exceed about 10° C. above the melting point of the fluid, preferably at a temperature that does not exceed about 5° C. above the melting point of the fluid. Thus, for example, when the biomolecule-containing fluid is aqueous, it may be optimal to keep the fluid at about 4° C. during ejection.

For some applications, especially those involving acoustic deposition of molten metals or other materials, a heating element may be provided for maintaining the substrate at a temperature below the melting point of the molten material, but above ambient temperature so that control of the rapidity of cooling may be effected. The rapidity of cooling may thus be controlled, to permit experimentation regarding the properties of combinatorial compositions such as molten deposited alloys cooled at different temperatures. For example, it is known that metastable materials are generally more likely to be formed from rapid cooling. The approach of generating materials by different cooling or quenching rates may be termed combinatorial quenching, which could be effected by changing the substrate temperature between acoustic ejections of the molten material. A more convenient method of evaluating combinatorial compositions solidified from the molten state at different rates is to generate multiple arrays, each of which has the same pattern of nominal compositions, but on substrates maintained at different temperatures.

It will be appreciated by those of ordinary skill in the art that the invention is also useful in the preparation of high-density combinatorial libraries containing a variety of synthetic, semi-synthetic, or naturally occurring moieties, because such libraries may require specifications that employ a large amount of memory. Such specifications may include instructions relating to various fabrication steps. It should be evident, then, that many variations of the invention are possible. For example, each of the ejected droplets may be deposited as an isolated and “final” feature, e.g., in spotting oligonucleotides, as mentioned above. Alternatively, or in addition, a plurality of ejected droplets may be deposited on the same location of a substrate surface in order to synthesize a biomolecular array in situ, as described above.

For array fabrication, it is expected that various washing steps may be used between droplet ejection steps. Such washing steps may involve, e.g., submerging the entire substrate surface on which features have been deposited in a washing fluid. In a modification of this process, the substrate surface may have deposited on it a fluid containing a reagent that chemically alters all features at substantially the same time, e.g., to activate and/or deprotect biomolecular features already deposited on the substrate surface to provide sites on which additional coupling reactions may occur.

In another embodiment, the invention relates to a device for performing an experiment using the moieties attached to the substrate surface of the inventive device. Such a device comprises a means for reading the machine-readable information contained in the substrate and a means for applying a substance that induces a response by the moieties. Optionally, the machine further comprises means for measuring the response and/or means for altering the information contained in, adding information to, or deleting information from, the substrate.

A method for performing an experiment using a plurality of moieties to a surface of a substrate may be performed by using the above device or another device. The method involves reading the information from the substrate and applying a substance that induces a response from the moieties based upon the information read by the machine. The information may be read by moving the substrate with respect to a reader. Such movement may involve rotating or laterally moving the substrate. In addition, the information may be read by converting the information contained in the substrate into electric current or light waves. Optimally, the same machine that reads the information from the substrate is used also to apply a substance that induces a response from the moieties based upon the information read by the machine. Such responses may be measured or detected, for example, in the form of fluorescence or radioactivity. Once the substance is applied to the attached moieties, the machine may be used to detect the response, and information relating to the response may be written on the substrate. For example, the attached moieties may represent an array of oligonucleotides adapted to screen for a particular nucleotidic sequence in a sample. In such a case, the sample containing labeled nucleotidic material may be applied to the attached moieties to determine whether any of the labeled nucleotidic material hybridizes with the surface-bound oligonucleotides. By detecting hybridization events, the screening experiment is performed. Information that describes the results of the experiment may be written on the substrate. As a result, the device may now contain all relevant information relating to the experiment from array formation to completion. Other experiments that involve using biomolecular arrays in conjunction with machine-readable data, for example, peptidic binding, may also be performed using the inventive devices and methods.

In general, screening for the properties of the array constituents will be performed in a manner appropriate to the type of array generated. Screening for biological properties such as ligand binding or hybridization may be generally performed in the manner described in U.S. Pat. Nos. 5,744,305 and 5,445,934 to Fodor et al. U.S. Pat. Nos. 5,143,854, 5,405,783 to Pirrung et al., and U.S. Pat. Nos. 5,700,637 and 6,054,270 to Southern et al. Routine methods for measuring physical and chemical properties may be easily adapted for screening material properties of the features of microarrays. In addition to bulk material characteristics or properties, surface specific properties may be measured by surface specific physical techniques and physical techniques that are adapted to surface characterization. Macroscopic surface phenomena, including adsorption, catalysis, surface reactions (including oxidation), hardness, lubrication, and friction, may be examined on a molecular scale using such characterization techniques. Various physical surface characterization techniques include, without limitation, diffractive techniques, spectroscopic techniques, microscopic surface imaging techniques, surface ionization mass spectroscopic techniques, thermal desorption techniques, and ellipsometry. It should be appreciated that these classifications are arbitrarily made for purposes of explication, and some overlap may exist.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains.

All patents, patent applications, journal articles, and other references cited herein are incorporated by reference in their entireties.

The following examples are put forth so as to provide those of ordinary skill in the art with a complete disclosure and description of how to implement the invention, and are not intended to limit the scope of what the inventors regard as their invention.

### EXAMPLE 1

This example describes construction of the inventive device comprising a substrate having a porous surface adapted for attachment to a plurality of moieties wherein the substrate contains machine-readable information relating to the attachment of the moieties to the substrate surface. The device is suitable for use in preparation of an array of biomolecular moieties in the form of a library.

A glass substrate is provided as a support, and microporous glass, preferably controlled pore size glass (CPG), is sintered onto a surface of the glass substrate to form a CPG layer having a thickness sufficient to enable permeation to both the downward flow and the lateral wicking of fluids. Generally, a sufficient thickness is greater than about 10 μm. Accordingly, the CPG is applied to the glass surface at a thickness of about 20 μm and the glass with powdered CPG resident thereon is heated at 750° C. for about 20 minutes and then cooled. Commercially available microscope slides (BDH Super Premium 76×26×1 mm) are used as supports. Depending on the specific glass slide and CPG material used, the sintering temperature and time may be adjusted to obtain a permeable and porous layer that is adequately attached to the glass beneath while substantially maintaining the permeability to fluids and thickness of the microporous glass layer. Slides heated for 20 minutes, with a 1 cm square patch of microporous glass applied at a pre-heating thickness of about 20 μm, yield a sintered layer of substantially the same depth as that present before heating, namely 20 μm.

Next, a microchip adapted to contain machine-readable information is attached to a substrate surface that opposes the surface on which CPG is applied. Attachment of the microchip after sintering allows the microchip to avoid the temperatures needed to carry out the sintering process. The chip is programmed with information associated with the synthesis of a nucleotidic array and instructions relating to the measurement of experimental results associated with the use of the nucleotidic array. The chip further contains a storage medium that allows information to be written therein relating to experimental results obtained using the device. A substrate is thus formed having a porous surface adapted for attachment to a plurality of moieties and containing machine-readable information relating to the moieties in a discrete region of the substrate, wherein the microchip represents the discrete region of the substrate.

### EXAMPLE 2

This example describes preparation of an array of oligonucleotides in the form of a library using the device generally described in Example 1, and demonstrates the use of focused acoustic energy in the solid phase synthesis of oligonucleotides.

The device formed in Example 1 is provided. As discussed above, the microchip is programmed with machine-readable information associated with the synthesis of a nucleotidic array. In this case, the machine-readable information relates to the instructions for preparing an oligonucleotidic library. Following the instructions contained in the chip, a machine is employed to derivatize the microporous glass layer of the substrate with a long aliphatic linker that can withstand conditions required to deprotect the aromatic heterocyclic bases, i.e. 30% NH₃ at 55° C. for 10 hours. The linker, which bears a hydroxyl moiety, the starting point for the sequential formation of the oligonucleotide from nucleotide precursors, is synthesized in two steps. First, the sintered microporous glass layer is treated for 20 hours at 90° C. with a 25% solution of 3-glycidoxypropyltriethoxysilane in xylene containing several drops of Hunig's base as a catalyst in a staining jar fitted with a drying tube. The slides are then washed with MeOH, Et₂O and air dried. Neat hexaethylene glycol and a trace amount of concentrated H₂SO₄ are then added and the mixture is kept at 80° C. for 20 hours. The slides are washed with MeOH, Et₂O, air dried, and stored desiccated at −20° C. until use. (This preparative technique is generally described in British Patent Application 8822228.6 filed Sep. 21, 1988.)

In addition, the machine-readable instructions provide for focused acoustic ejection of about 0.24 pL of anhydrous acetonitrile (the primary coupling solvent) containing a fluorescent marker onto the microporous substrate. As a result, a circular patch of about 5.6 μm diameter is formed on the permeable sintered microporous glass substrate. The amount of acoustic energy applied at the fluid surface may be adjusted to ensure an appropriate diameter of chemical synthesis for the desired site density. Circular patches 5.6 μm in diameter are suitable for preparing an array having a site density of 10⁶ sites/cm², with the circular synthetic patches spaced 10 μm apart center to center, and the synthetic patches therefore spaced edge to edge at least 4 μm apart at the region of closest proximity. All subsequent spatially directed acoustically ejected volumes in this example are of about 0.24 pL; it will be readily appreciated that the ejection volumes can be adjusted for solutions other than pure acetonitrile by adjusting the acoustic energy as necessary for delivery of an appropriately sized droplet after spreading on the substrate (here about a 5 μm radius).

The instructions also direct the machine to carry out an oligonucleotide synthesis cycle. A coupling solution is prepared by mixing equal volumes of 0.5M tetrazole in anhydrous acetonitrile and a 0.2M solution of the required β-cyanoethylphosphoramidite, e.g. A-β-cyanoethylphosphoramidite, C-β-cyanoethylphosphoramidite, G-β-cyanoethylphosphoramidite, T (or U)-β-cyanoethylphosphoramidite. Coupling time is three minutes. Oxidation with a 0.1M solution of I₂ in THF/pyridine/H₂O yields a stable phosphotriester bond. Detritylation of the 5′ end with 3% trichloroacetic acid (TCA) in dichloromethane allows further extension of the oligonucleotide chain. No capping step is required because the excess of phosphoramidites used over reactive sites on the substrate is large enough to drive coupling to completion. After coupling the slide the subsequent chemical reactions (oxidation with I₂, and detritylation by TCA) are performed by dipping the slide into staining jars. Alternatively, the focused acoustic delivery of I₂ in THF/pyridine/H₂O and/or 3% TCA in dichloromethane to effect the oxidation and tritylation steps only at selected sites may be performed if sufficient time transpires to permit evaporation of substantially all the solvent from the previous step so that the synthetic patch edges do not move outwards and closer to the neighboring synthetic patches, and further to provide an anhydrous environment for subsequent coupling steps if I₂ in THF/pyridine/H₂O is delivered within the reaction chamber.

After the synthesis is complete, the oligonucleotide is deprotected in 30% NH₃ for 10 hours at 55° C. Because the coupling reagents are moisture-sensitive, the coupling step must be performed under anhydrous conditions in a sealed chamber or container. This may be accomplished by performing the acoustic spotting in a chamber of desiccated gas obtained by evacuating a chamber that contains the acoustic ejection device and synthetic substrate and replacing the evacuated atmospheric gas with desiccated N₂ by routine methods; washing steps may be performed in the chamber by removing the slide and washing it in an appropriate environment, for example, in a staining jar fitted with a drying tube. Because washing and other steps such as detritylation may be more conveniently achieved outside the chamber, the synthesis may also be performed in a controlled humidity room that contains the controlled atmosphere chamber in which the spotting is done, with the other steps carried out in the room outside the chamber. Alternatively, a controlled humidity room may be used for spotting with the other steps carried out in a less controlled environment by use of, for example, a staining jar fitted with a drying tube.

### EXAMPLE 3

This example describes the use of an array of oligonucleotides formed in Example 2 as probes in a hybridization experiment, and demonstrates the use of a machine to screen for a particular nucleotidic sequence in a sample.

The array of oligonucleotides formed in Example 2 is provided. The attached oligonucleotides serve as probes to assess whether target nucleotidic moieties are present in a sample. About 20 μl of sample fluid comprising a buffered aqueous solution containing fluorescently labeled target mRNA is placed on the substrate surface to which oligonucleotides are attached. By spreading the target solution over the array with a cover, every part of the probe-containing surface is provided with substantially equivalent exposure to the target solution to ensure uniform hybridization conditions at each probe. The cover in combination with the substrate represents a hybridization assembly. The assembly is then subjected to the hybridization conditions. Although the assembly is substantially sealed, the assembly may be brought to the hybridization temperature in a humidified environment to further lower the possibility of target solution evaporation during the hybridization reaction. The hybridization reaction typically takes place over a time period of as much as many hours.

After hybridization is complete, the substrate is washed to remove mRNA that has not been hybridized to an attached probe. A detector is operated according to instructions contained in the microchip to determine the quantity of mRNA hybridized at each probe location using a fluorescence detector. As a result, the screening experiment is performed. That is, by determining whether mRNA has been hybridized to the attached probes and the extent of hybridization (if any), the sample is screened for complementary sequences to the attached probes. Information that describes the results of the experiment is written on the microchip. Accordingly, the device now contains all relevant information relating to the experiment from array formation to completion.

We claim:

1. A device comprising a substrate having a plurality of molecular moieties attached to a surface thereof and containing machine-readable information relating to the molecular moieties, wherein the information is contained in a discrete region of the substrate that is non-coplanar with respect to the substrate surface having the plurality of molecular moieties attached thereto.

2. The device of claim 1, wherein the machine-readable information comprises the identity of a customer.

3. The device of claim 1, wherein the machine-readable information comprises secured information.

4. The device of claim 1, wherein the machine-readable information comprises shipping and/or billing information.

5. The device of claim 1, wherein the machine-readable information comprises the identity of at least one of the molecular moieties attached to the device surface.

6. The device of claim 1, wherein the machine-readable information comprises information relating to a process by which the plurality of molecular moieties is attached to the substrate surface.

7. The device of claim 1, wherein the machine-readable information comprises information relating to experimental conditions associated with a use of the plurality of molecular moieties.

8. The device of claim 1, wherein the machine-readable information comprises information relating to the results of an experiment associated with a use of the plurality of molecular moieties.

9. The device of claim 1, wherein the machine-readable information is digital.

10. The device of claim 9, wherein the machine-readable information is represented by no less than about 1 kilobyte of data.

11. The device of claim 10, wherein the machine-readable information is represented by no less than about 1 megabyte of data.

12. The device of claim 11, wherein the machine-readable information is represented by about 1 to about 650 megabytes of data.

13. The device of claim 1, wherein the machine-readable information is optically readable.

14. The device of claim 13, wherein the machine-readable information is readable by a fluorescence reader.

15. The device of claim 13, wherein the machine-readable information is readable by a phosphoimager (i.e. can detect radioactive signal produced on sensitive film).

16. The device of claim 13, wherein the machine-readable information is readable by a compact disk reader.

17. The device of claim 13, wherein the machine-readable information is readable by a DVD reader.

18. The device of claim 13, wherein the machine-readable information is readable by a bar code reader.

19. The device of claim 18, wherein the bar code reader is a one-dimensional bar code reader.

20. The device of claim 18, wherein the bar code reader is a two-dimensional bar code reader.

21. The device of claim 1, wherein the machine-readable information is magnetically readable.

22. The device of claim 1, wherein the machine-readable information is electronically readable.

23. The device of claim 1, further comprising human readable information.

24. The device of claim 1, wherein the attached molecular moieties are protected.

25. The device of claim 24, further comprising a protective layer over the attached molecular moieties.

26. The device of claim 25, wherein the protective layer is removable.

27. The device of claim 25, wherein the protective layer allows only selected matter to be transmitted therethrough.

28. The device of claim 27, wherein the selected matter is electromagnetic radiation.

29. The device of claim 28, wherein the electromagnetic radiation has a wavelength that causes fluorescence near an attached molecular moiety.

30. The device of claim 1, wherein the plurality of attached molecular moieties comprises an array of biomolecules.

31. The device of claim 30, wherein the biomolecules are nucleotidic or peptidic.

32. The device of claim 30, wherein the biomolecules are oligomeric or polymeric.

33 The device of claim 30, wherein the array comprises at least about 5,000 molecular moieties per square centimeter of substrate surface.

34. The device of claim 33, wherein the array comprises at least about 50,000 molecular moieties per square centimeter of substrate surface.

35. The device of claim 34, wherein the array comprises at least about 200,000 molecular moieties per square centimeter of substrate surface.

36. The device of claim 35, wherein the array comprises at least about 1,000,000 molecular moieties per square centimeters of substrate surface.

37. The device of claim 1, wherein the substrate comprises a disk.

38. The device of claim 1, wherein the substrate comprises a tape.

39. The device of claim 1, wherein the substrate comprises a well plate.

40. The device of claim 1, wherein the substrate comprises a slide.

41. The device of claim 1, wherein the substrate comprises a plurality of surfaces arranged in a three-dimensional structure to which the molecular moieties are attached

42. The device of claim 1, wherein the substrate comprises a magnetic medium on which additional information may be written.

43. The device of claim 1, wherein the substrate comprises an optical medium on which additional information may be written.

44. The device of claim 1, wherein the surface having the molecular moieties attached thereto opposes a surface on which the information is located.

45. A device comprising a substrate having a surface adapted for attachment to a plurality of molecular moieties and containing machine-readable information relating to the molecular moieties.

46. The device of claim 45, wherein the machine-readable information is located on a surface of the substrate that is non-coplanar with respect to the surface adapted for attachment to a plurality of molecular moieties.

47. The device of claim 45, wherein attachment of molecular moieties to the surface is detectable through a signal having the same form as the machine-readable information.

48. The device of claim 47, wherein the signal form is fluorescence.

49. The device of claim 47, wherein the signal form is radioactivitiy.

50. The device of claim 46, wherein the non-coplanar surface opposes the surface adapted for attachment to a plurality of molecular moieties.

51. A machine for attaching molecular moieties to a device comprising a substrate having a surface adapted for attachment to a plurality of molecular moieties and containing machine-readable information relating to the molecular moieties, comprising:
a reader for reading the machine-readable information from the device; and
a means for attaching a plurality of biomolecules to the surface of the substrate based upon the machine readable information contained in the substrate.

52. The machine of claim 51, further comprising a means for verifying attachment of the biomolecules to the surface of the substrate.

53. The machine of claim 52, wherein the means for verifying attachment of the biomolecules is the reader for reading the machine-readable information.

54. The machine of claim 53, wherein the reader is adapted to detect fluorescence.

55. The machine of claim 53, wherein the reader is adapted to detect phosphorescence.

56. The machine of claim 5 1, wherein the attaching means comprises:
a reservoir adapted to contain a fluid;
an acoustic radiation generator for generating acoustic radiation; and
a focusing means for focusing the acoustic radiation at a focal point near the fluid surface in the reservoir.

57. The machine of claim 51, further comprising a means for altering the machine-readable information in the substrate.

58. A machine for performing an experiment using a device comprising a substrate having a plurality of molecular moieties attached to a surface thereof and containing machine-readable information relating to the molecular moieties, wherein the information is contained in a discrete region of the substrate that is non-coplanar with respect to the substrate surface having the plurality of molecular moieties attached thereto, comprising:
a reader for reading the machine-readable information contained in the device; and
a means for applying a substance that induces a response by the molecular moieties.

59. The machine of claim 58, further comprising a means for measuring the response.

60. The machine of claim 59, wherein the means for measuring the response is the reader for reading the machine-readable information.

61. The machine of claim 60, wherein the response is fluorescence.

62. The machine of claim 60, wherein the response is phosphorescence.

63. The machine of claim 58, further comprising a means for altering the machine-readable information in the substrate.

64. A method for attaching a plurality of molecular moieties to a surface of a substrate, comprising the steps of:
(a) providing the device of claim 45;
(b) using a machine to read the information from the substrate;
(c) attaching a plurality of molecular moieties to a surface of the substrate according to the information read by the machine.

65. The method of claim 64, wherein step (b) comprises the step of moving the substrate with respect to the machine.

66. The method of claim 65, wherein step (b) comprises determining the position and/or orientation of the substrate with respect to the machine.

67. The method of claim 65, wherein the moving step involves rotating the substrate.

68. The method of claim 65, wherein the moving step involves laterally moving the substrate.

69. The method of claim 65, wherein step (b) comprises the step of converting the information contained in the substrate into electric current.

70. The method of claim 65, wherein step (b) comprises the step of converting the information contained in the substrate into light waves.

71. The method of claim 65, wherein step (c) comprises the step of ejecting fluid droplets on the surface.

72. The method of claim 71, wherein the ejecting step is carried out acoustically.

73. The method of claim 72, wherein the ejecting step is carried out without using a nozzle.

74. The method of claim 64, wherein step (c) comprises attaching no more than one biomolecule at a time.

75. The method of claim 64, wherein step (c) comprises using a photolithographic technique.

76. The method of claim 64, wherein step (c) comprises covalently attaching the molecular moieties to the substrate surface.

77. The method of claim 64, wherein step (c) comprises noncovalently attaching the molecular moieties to the substrate surface.

78. The method of claim 64, wherein step (c) is performed by the machine that reads information from the substrate.

79. The method of claim 64, wherein step (c) comprises lowering the temperature of the substrate.

80. The method of claim 64, wherein steps (b) and (c) are performed substantially simultaneously.

81. The method of claim 64, wherein steps (b) and (c) are alternatingly repeated.

82. A method for performing an experiment using a plurality of molecular moieties attached to a surface of a substrate, comprising the steps of:
(a) providing a device of claim 1;
(b) using a machine to read the information from the substrate;
(c) applying a substance that induces a response from the molecular moieties based upon the information read by the machine.

83. The method of claim 82, wherein step (b) comprises the step of moving the substrate with respect to the machine.

84. The method of claim 83, wherein the moving step involves rotating the substrate.

85. The method of claim 83, wherein the moving step involves laterally moving the substrate.

86. The method of claim 82, wherein step (b) comprises the step of converting the information contained in the substrate into electric current.

87. The method of claim 82, wherein step (b) comprises the step of converting the information contained in the substrate into light waves.

88. The method of claim 82, wherein step (c) is performed by the machine that reads information from the substrate.

89. The method of claim 82, further comprising step (d) detecting the response.

90. The method of claim 89, further comprising step (e) writing information relating to the response on the substrate.
